(19) United States
(12) Patent Application Publication
(10) Pub. No.: US 2004/0181255 A1
(43) Pub. Date: Sep. 16, 2004

(54) Disposable tweezers with built-in illumination source for personal hygiene, cosmetic and medical applications

(76) Inventor: Harry Gio, Niles, IL (US)

Correspondence Address: Harry Gio, 9938 N. Huber Lane, Niles, IL, 60714, US

(21) Appl. No.: 10/385,887

(22) Filed: Mar. 12, 2003

(51) Int. Cl. A61B 17/50

(52) U.S. Cl. 606/210

(57) ABSTRACT

Seezers is a system combining standard stainless steel tweezers, a white LED, and an enveloping coating to completely seal all wiring and power sources from external exposure. The use of a white LED to aid in the removal process of foreign objects from human skin is unique and the primary distinguishing characteristic of Seezers. Seezers exterior protective coating is designed to accommodate a variety of colored dyes and machine textures for user customization.

### CROSS REFERENCE TO RELATED APPLICATIONS

Not applicable

### STATEMENT REGARDING FEDERALLY SPONSORED RESEARCH OR DEVELOPMENT

Not applicable

### REFERENCE TO SEQUENCE LISTING, A TABLE, OR A COMPUTER PROGRAM LISTING COMPACT DISK APPENDIX

Not applicable

### FIELD OF INVENTION

The invention, disposable tweezers with built-in illumination source for personal hygiene, cosmetic and medical application, is relevant to and intended by and expected by its inventor to be fully utilized within the current and future fields of personal hygiene management, commercial cosmetic application and preparation and a wide range of medical fields including, but not limited to surgery and basic first aid implementation

### BRIEF SUMMARY OF THE INVENTION

The invention, disposable tweezers with built-in illumination source for personal hygiene, Cosmetic and medical application, combines the utility of a specially-gripped tweezers with a high-power illumination system to allow the user to clearly see any object being removed or placed by the gripping tips of the tweezers. The inclusion of the illumination system solves the problem of not being able to clearly see objects being manipulated with the invention. The inclusion of the illumination system allows users with poor eyesight to more clearly identify areas of skin for removal or placement of objects. The primary objective of the invention is to allow the clear removal or placement of objects primarily in or on the skin.

### BRIEF DESCRIPTION OF THE SEVERAL VIEWS OF THE DRAWING

FIG. 1—Side cross section view of invention illustrating internal materials

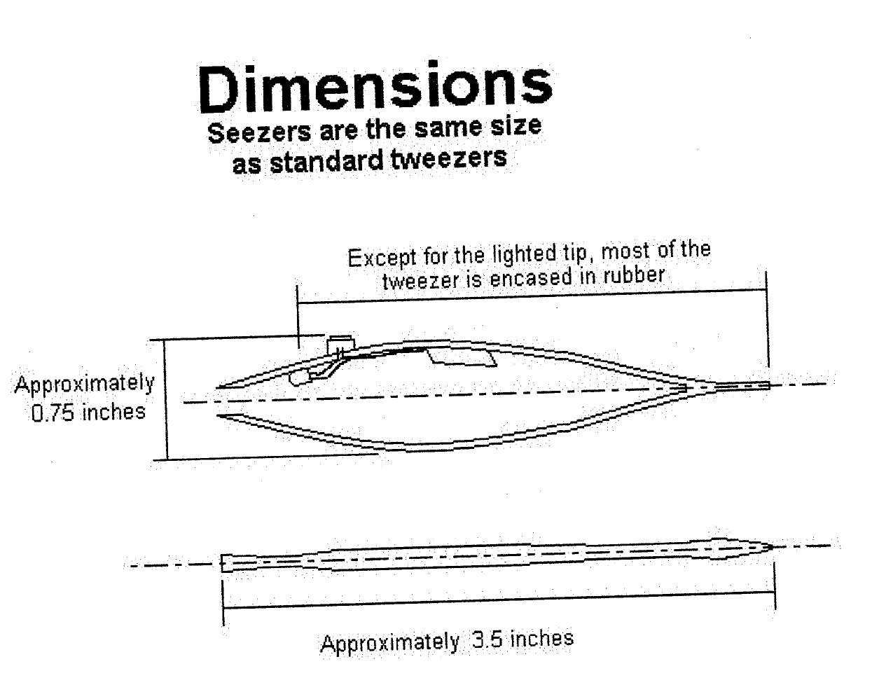

### DETAILED DESCRIPTION OF THE INVENTION

The invention, disposable tweezers with built-in illumination source for personal hygiene, cosmetic and medical application, consists of two systems, the Illumination System and the Insulating System. The Illumination System consists of (1) insulated copper electrical wiring, (2) one white LED, (3) two small watch or calculator batteries and (4) a thumb switch for turning on and off the LED. The Gripping System consists of a rubber coating which covers the entire invention except for the LED, on/off switch, and the tips of the tweezers, themselves. The Illumination System is ally housed and contained within the Insulating System such that no part of the Illumination System can be accessed without partial or full destruction of the invention, with the sole exception of the thumb switch for turning on and off the LED. The LED is mounted underneath the upper tweezer arm, The thumb switch for turning on and off the light bulbs is located on the upper pincer-like arm approximately two inches from the tip of the pincer-like arm. The watch/calculator batteries are located directly under the thumb switch. The insulated copper electrical wiring runs the length of upper pincer-like arm from the thumb switch to the LED.

The Insulating System is made from rubber coating that shrinks as it cools to encase the invention excluding the LED and the tips of the tweezer arms.

The thumb switch allows the user to (at will) engage and disengage the illumination source (LED) as necessary. The invention is designed and intended to be disposable so as to increase the level of personal hygiene and meet medical sterility requirements.

1. What I claim as my invention is a hand-held and hand-operated instrument for removing or placing an infinite variety of objects and materials from and/or onto human skin or any other relevant surface or material.

2. What I claim as my invention is a hand-held and hand-operated instrument made of steel partially sealed within a rubber coating so as to render it inoperable should the instrument be forced open.

3. What I claim as my invention is a hand-held and hand-operated instrument resembling a common set of tweezers covered in a rubber coating.

4. What I claim as my invention is a hand-held and hand-operated instrument resembling a common pair of tweezers with one single white LED mounted between the upper and lower tweezer arms approximately 1 inch from the tweezer tips.

5. What I claim as my invention is a hand-held and hand-operated instrument resembling a common pair of tweezers which has a common thumb-operated electrical switch located on the end of the twezer boated where the tweezer arms join together.

6. What I claim as my invention is a hand-held and hand-operated instrument resembling a common pair of tweezers with pincer tips shaped into a combination of flat tipped and needle-nose tipped ends to afford the benefits of both designs for gripping and pulling capabilities.

7. What I claim as my invention is a hand-held and hand-operated instrument resembling a common pair of tweezers which contains an internal power source of five hearing aid batteries used to operate the single white LED whose power source is connected to the LED and thumb-operated electrical switch by insulated copper electrical wire and whose entire illumination system and power source and encased completely in a rubber coating.

8. What I claim as my invention is a hand-held and hand-operated instrument resembling a common pair of tweezers whose entire unit, except for the illumination system and power source, is made of steel partially covered in a rubber coating designed to remain closed to the environment to make the instrument disposable.

9. What I claim as my invention is a hand-held and hand-operated instrument resembling a common pair of tweezers whose entire unit, except for the illumination system and power source, is made of high-impact plastic designed to be capable of being covered with a variety of colors so as to allow for aesthetic variations for marketing purposes only.
